(19) United States
(12) Patent Application Publication
(10) Pub. No.: US 2011/0137102 A1
(43) Pub. Date: Jun. 9, 2011

(54) STEREOTACTIC INTRACRANIAL TARGET LOCALIZATION GUIDANCE SYSTEMS AND METHODS

(75) Inventor: Siyong KIM, Saint Johns, FL (US)

(73) Assignee: Mayo Foundation for Medical Education and Research, Rochester, MN (US)

(21) Appl. No.: 12/794,201

(22) Filed: Jun. 4, 2010

Related U.S. Application Data
(60) Provisional application No. 61/184,154, filed on Jun. 4, 2009.

(51) Int. Cl. A61N 5/00

(52) U.S. Cl. 600/1

(57) ABSTRACT

Systems and methods of intracranial target localization using non-radiological direct surface imaging of one or more of the upper teeth.

### RELATED APPLICATION

This application claims the benefit under 35 U.S.C. §119 of U.S. Provisional Application Ser. No. 61/184,154, filed Jun. 4, 2009, entitled STEREOTACTIC INTRACRANIAL TARGET LOCALIZATION GUIDANCE SYSTEMS AND METHODS, which is incorporated herein by reference in its entirety.

Systems and methods for frameless stereotactic intracranial target localization guidance are described herein.

The systems and methods described herein may be used for intracranial guidance and localization based on non-radiological real-time image guidance for any purpose including, but not limited to, stereotactic radiosurgery (SRS), stereotactic radiotherapy, etc.

Intracranial stereotactic radiosurgery (SRS) treats brain disorders with a precise delivery of a single, high dose of radiation in a one-day session. Stereotactic radiotherapy (SRT) treats brain disorders with one or more doses of radiation that are fractionated over selected time periods (e.g., hours, days, etc.). Focused radiation beams are delivered to a specific area of the brain to treat abnormalities, tumors or functional disorders.

One method of target localization is to use a metal head frame that is invasively attached to the subject's skull. Other methods have involved the use of bite plates in which a dental impression is used to construct a bite plate. Markers are attached to a frame that is mounted on the bite plate and used to determine target location using radiological imaging processes. This system does not, however, provide for verification of secure and reproducible contact between the upper teeth and the dental impression.

The use of radiological imaging (e.g., CT imaging) alone to identify the location of targeted tissue relative to a subject's teeth is not reliable because it is not easy to get accurate information of the relationship between the upper teeth and the targeted tissue in CT images because of inherent limitations of CT imaging in obtaining precise images of the teeth and possible image artifacts near the teeth.

The following documents may describe a variety of systems and methods that may be used for stereotactic target localization and/or radiosurgery/radiotherapy and which may potentially be adapted to use the non-radiological image guidance systems and methods described herein: U.S. Pat. No. 5,207,223 (Adler); U.S. Pat. No. 5,427,097 (Depp); U.S. Pat. No. 5,528,651 (Leksell et al.); U.S. Pat. No. 6,778,850 (Adler et al.); U.S. Pat. No. 6,876,196 (Taulu et al.); U.S. Pat. No. 7,348,974 (Smith et al.); etc.

### SUMMARY

The systems and methods described herein may, in some embodiments, improve the accuracy of intracranial target localization without requiring an invasively attached head frame and reducing the need for radiological imaging. The systems and methods of some embodiments described herein use direct surface imaging of one or more of the upper teeth for localization of intracranial targets. The upper teeth are rigidly connected to the skull and, therefore, can be used to determine the position of any intracranial target in a manner that is both accurate and repeatable. The systems and methods may also potentially provide real time image-guidance through the radiation delivery process.

In still other systems and methods described herein, non-radiological imaging may be used to determine the position of a head ring attached to a subject's head relative to a tooth to provide a mechanism for verifying that the position of the head ring has not shifted over time.

In one aspect, some embodiments of a stereotactic intracranial target localization guidance system as described herein includes: a subject support device comprising a head immobilizer; a primary imaging apparatus, wherein the primary imaging apparatus comprises a non-radiological imaging system; a reference cage defining a three-dimensional volume, wherein the reference cage comprises radio-opaque material; and a controller operably connected to the primary imaging apparatus to receive direct image data from the primary imaging apparatus and wherein the controller receives radiological image data from a radiological imaging apparatus. The controller is configured to: determine the location of targeted tissue relative to the reference cage using the radiological image data from the radiological imaging apparatus; determine the location of a selected upper tooth of a subject positioned in the subject support device relative to the reference cage using the direct image data from the primary imaging apparatus; and determine the location of the targeted tissue relative to the selected upper tooth using the determined location of targeted tissue relative to the reference cage and the determined location of the selected upper tooth relative to the reference cage.

In some embodiments of the systems described herein, the reference cage is not attached to a mouth opening device.

In some embodiments of the systems described herein, the reference cage is fixedly attached to the subject support device.

In some embodiments of the systems described herein, the head immobilizer comprises an inflatable helmet.

In some embodiments of the systems described herein, the primary imaging apparatus comprises a stereographic imaging system.

In some embodiments of the systems described herein, the controller is operably connected to a radiation beam generator, wherein the controller is configured to direct the radiation beam generator to emit a beam of radiation at the targeted tissue based on the location of the tooth.

In some embodiments of the systems described herein, the subject support comprises a translational mechanism and/or rotational mechanism, and wherein the controller is operably connected to the translational mechanism and/or rotational mechanism of the subject support, and further wherein the controller is configured to operate the translational mechanism and/or rotational mechanism to position the determined location of the targeted tissue in a predetermined location.

In some embodiments of the systems described herein, the system comprises an auxiliary imaging apparatus located in a fixed position within a treatment room, wherein the auxiliary imaging apparatus obtains a non-radiological auxiliary image of the primary imaging apparatus, wherein the auxiliary imaging apparatus is operably connected to the controller, and wherein the controller is configured to: determine the location of the primary imaging apparatus in the treatment room using auxiliary image data from the auxiliary imaging apparatus; and determine the location of the targeted tissue within the treatment room using the determined location of the primary imaging apparatus.

In some embodiments of systems including an auxiliary imaging apparatus, the auxiliary imaging apparatus comprises a stereographic imaging system.

In some embodiments of systems including an auxiliary imaging apparatus, the controller is operably connected to a radiation beam generator, wherein the controller is configured to direct the radiation beam generator to emit a beam of radiation at the determined location of the targeted tissue.

In some embodiments of systems including an auxiliary imaging apparatus, the subject support comprises a translational mechanism and/or rotational mechanism, and wherein the controller is operably connected to the translational mechanism and/or rotational mechanism of the subject support, and further wherein the controller is configured to operate the translational mechanism and/or rotational mechanism to position the determined location of the targeted tissue in a predetermined location.

In a second aspect, some embodiments of methods for determining the location of targeted tissue relative to a tooth, the method includes: positioning a head of a subject in a volume defined by an reference cage; exposing a selected upper tooth of the subject while the head is positioned in the volume; obtaining a direct image of the selected upper tooth using a non-radiological primary imaging apparatus; obtaining a radiological image that includes the targeted tissue and the reference cage; restraining the head of the subject in a fixed position relative to the reference cage while obtaining the direct image of the selected upper tooth using the primary imaging apparatus and while obtaining the radiological image that includes the targeted tissue and the reference cage; determining the location of targeted tissue in the head relative to the reference cage using the radiological image; determining the location of the selected upper tooth relative to the reference cage using the direct image from the primary imaging apparatus; and determining the location of the targeted tissue relative to the selected upper tooth using the location of targeted tissue relative to the reference cage and the location of the selected upper tooth relative to the reference cage.

In some embodiments, methods according to the second aspect described herein may include exposing the selected upper tooth comprises placing a mouth opening device in the mouth of the subject.

In some embodiments, methods according to the second aspect described herein may include a mouth opening device that is not fixedly attached to the reference cage.

In some embodiments, methods according to the second aspect described herein may include fixedly attaching the reference cage to the subject support device.

In some embodiments, methods according to the second aspect described herein may include restraining the head of the subject in a fixed position by positioning the head in an inflatable helmet and inflating the helmet.

In some embodiments, methods according to the second aspect described herein may include using a primary imaging apparatus that comprises a stereographic imaging system.

In some embodiments, methods according to the second aspect described herein may include: obtaining an auxiliary image of the primary imaging apparatus using non-radiological auxiliary imaging apparatus located in a fixed position within a treatment room in which the reference cage is located; determining the location of the primary imaging apparatus in the treatment room using auxiliary image data from the auxiliary imaging apparatus; and determining the location of the targeted tissue within the treatment room based using the determined location of the primary imaging apparatus. In some embodiments, the auxiliary imaging apparatus comprises a stereographic imaging system.

In some embodiments, methods according to the second aspect that include obtaining an auxiliary image may include adjusting the position of the subject support to place the targeted tissue in a predetermined location within the treatment room.

In some embodiments, methods according to the second aspect that include obtaining an auxiliary image may include emitting a beam of radiation at the determined location of the targeted tissue.

In some embodiments, methods according to the second aspect described herein may include adjusting the position of the subject support to place the targeted tissue in a predetermined location within a treatment room.

In some embodiments, methods according to the second aspect described herein may include emitting a beam of radiation at the determined location of the targeted tissue.

In a third aspect, some embodiments of the stereotactic intracranial target localization guidance systems described herein may include: a primary imaging apparatus, wherein the primary imaging apparatus comprises a non-radiological imaging system; a stereotactic head ring defining a three-dimensional volume proximate a subject's head, wherein the head ring comprises mechanisms for attaching the head ring directly to the subject, and wherein the head ring comprises radio-opaque material; and a controller operably connected to the primary imaging apparatus to receive direct image data from the primary imaging apparatus, wherein the controller is configured to determine an original location of the head ring relative to the tooth using the direct image data from the primary imaging apparatus.

In some embodiments of systems according to the third aspect, the system further comprises a subject support device that optionally comprises a head immobilizer.

In a fourth aspect, some embodiments of methods for determining a location of a head ring relative to a tooth as described herein may include: attaching a stereotactic head ring to a head of a subject; exposing a selected upper tooth of the subject while the stereotactic head ring is attached to the head; obtaining a direct image of the selected upper tooth and the head ring using a non-radiological primary imaging apparatus; and determining an original location of the selected upper tooth relative to the head ring using the direct image from the primary imaging apparatus.

In some embodiments, methods according to the fourth aspect described herein may include determining the location of targeted tissue in the head of the subject relative to the head ring using a radiological imaging system after determining the original location of the selected upper tooth relative to the head ring using the primary imaging apparatus.

In some embodiments, methods according to the fourth aspect described herein may include adjusting the position of the head ring on the head of the subject such that the head ring and the selected upper tooth are in the original position.

In some embodiments, a stereotactic intracranial target localization guidance system described herein may include a subject support device having a head immobilizer; a primary imaging apparatus, wherein the primary imaging apparatus is a non-radiological imaging system; a mouth opening device, wherein the primary imaging apparatus obtains a direct image of a tooth through the mouth opening device when the mouth opening device is in place within the mouth of a subject; and a reference cage defining a three-dimensional volume, wherein the reference cage is not attached to the mouth opening device, wherein the reference cage includes radio-opaque material.

In further embodiments, the systems described herein may include a controller operably connected to the primary imaging apparatus to receive direct image data from the primary imaging apparatus and wherein the controller receives radiological image data from a radiological imaging apparatus, wherein the controller further: determines the location of targeted tissue relative to the reference cage using the radiological image data from the radiological imaging apparatus; determines the location of the tooth relative to the reference cage using the direct image data from the primary imaging apparatus; and determines the location of the targeted tissue relative to the tooth using the determined location of targeted tissue relative to the reference cage and the determined location of the tooth relative to the reference cage.

In some embodiments, the controller is operably connected to a radiation beam generator, and the controller directs the radiation beam generator to emit a beam of radiation at the targeted tissue based on the location of the tooth.

In some embodiments, the subject support includes a translational mechanism and/or rotational mechanism, and the controller is operably connected to the translational mechanism and/or rotational mechanism of the subject support, and further wherein the controller operates the translational mechanism and/or rotational mechanism to position the determined location of the targeted tissue in a predetermined location.

In some embodiments, the system includes an auxiliary imaging apparatus located in a fixed position within a treatment room, wherein the auxiliary imaging apparatus obtains a non-radiological auxiliary image of the primary imaging apparatus, and wherein the auxiliary imaging apparatus is operably connected to the controller. The controller determines the location of the primary imaging apparatus in the treatment room using auxiliary image data from the auxiliary imaging apparatus; and determines the location of the targeted tissue within the treatment room using the determined location of the primary imaging apparatus. In still further embodiments, the auxiliary imaging apparatus is a stereographic imaging system. In still other embodiments, the controller is operably connected to a radiation beam generator, wherein the controller directs the radiation beam generator to emit a beam of radiation at the determined location of the targeted tissue. In still other embodiments, the subject support includes a translational mechanism and/or rotational mechanism, and wherein the controller is operably connected to the translational mechanism and/or rotational mechanism of the subject support, and the controller operates the translational mechanism and/or rotational mechanism to position the determined location of the targeted tissue in a predetermined location.

In some embodiments, the mouth opening device includes a frame defining a central opening; a lower tooth channel; a left side upper tooth channel; a right side upper tooth channel; and an upper lip restraint raising the upper lip in the superior direction. When the mouth opening device is in place in a mouth, the front teeth between the left side upper tooth channel and the right side upper tooth channel are exposed for imaging by the primary imaging apparatus.

In some embodiments, the mouth opening device is constructed of radio-lucent material.

In some embodiments, a method of determining the location of targeted tissue relative to a tooth as described herein may include positioning a head of a subject in a volume defined by an reference cage, wherein the reference cage is not directly attached to the subject's head; exposing a tooth of the subject while the head is positioned in the volume; obtaining a direct image of the tooth using a non-radiological primary imaging apparatus; obtaining a radiological image that includes the targeted tissue and the reference cage; restraining the head of the subject in a fixed position relative to the reference cage while obtaining the direct image of the tooth using the primary imaging apparatus and while obtaining the radiological image that includes the targeted tissue and the reference cage; determining the location of targeted tissue in the head relative to the reference cage using the radiological image; determining the location of the tooth relative to the reference cage using the direct image from the primary imaging apparatus; and determining the location of the targeted tissue relative to the tooth using the location of targeted tissue relative to the reference cage and the location of the tooth relative to the reference cage.

In some embodiments of the methods described herein, exposing the tooth may include placing a mouth opening device in the mouth of the subject, wherein the mouth opening device has a frame defining a central opening, a lower tooth channel, a left side upper tooth channel, a right side upper tooth channel, and an upper lip restraint; and wherein the upper lip restraint raises the upper lip in the superior direction such that the tooth is exposed between the left side upper tooth channel and the right side upper tooth channel. In some embodiments, the mouth opening device is constructed of radio-lucent material.

In some embodiments of the methods described herein, the method further includes obtaining an auxiliary image of the primary imaging apparatus using a non-radiological auxiliary imaging apparatus located in a fixed position within a treatment room in which the reference cage is located; determining the location of the primary imaging apparatus in the treatment room using auxiliary image data from the auxiliary imaging apparatus; and determining the location of the targeted tissue within the treatment room based using the determined location of the primary imaging apparatus. In still other embodiments, the auxiliary imaging apparatus is a stereographic imaging system. In still other embodiments, the method includes adjusting the position of the subject support to place the targeted tissue in a predetermined location within the treatment room. In still other embodiments, the method includes directing the radiation beam generator to emit a beam of radiation at the determined location of the targeted tissue.

In some embodiments of the methods described herein, the method further includes adjusting the position of the subject support to place the targeted tissue in a predetermined location within a treatment room.

In some embodiments of the methods described herein, the method further includes directing a radiation beam generator to emit a beam of radiation at the determined location of the targeted tissue.

In some embodiments, a stereotactic intracranial target localization guidance system described herein includes an optional subject support device and/or an optional head immobilizer; a primary imaging apparatus, wherein the primary imaging apparatus is a non-radiological imaging system; a mouth opening device, wherein the primary imaging apparatus obtains a direct image of a tooth through the mouth opening device when the mouth opening device is in place within the mouth of a subject; and a stereotactic head ring defining a three-dimensional volume proximate a subject's head, wherein the head ring includes mechanisms for attaching the head ring directly to the subject, and wherein the head ring includes radio-opaque material.

In some embodiments, the stereotactic intracranial target localization guidance system described herein includes a controller operably connected to the primary imaging apparatus to receive direct image data from the primary imaging apparatus, wherein the controller further determines an original location of the head ring relative to the tooth using the direct image data from the primary imaging apparatus.

In some embodiments, a method of determining location of a head ring relative to a tooth is described herein, the method including attaching a stereotactic head ring to a head of a subject; exposing a tooth of the subject while the stereotactic head ring is attached to the head; obtaining a direct image of the tooth and the head ring using a non-radiological primary imaging apparatus; and determining an original location of the tooth relative to the head ring using the direct image from the primary imaging apparatus.

In some embodiments, the method described in the preceding paragraph may include determining the location of targeted tissue in the head of the subject relative to the head ring using a radiological imaging system after determining the original location of the tooth relative to the head ring using the primary imaging apparatus.

In some embodiments, the method described in either of the preceding paragraphs may include adjusting the position of the head ring on the head of the subject such that the head ring and the tooth are in the original position.

The above summary is not intended to describe each embodiment or every implementation of the systems and methods described herein. Rather, a more complete understanding of the systems and methods will become apparent and appreciated by reference to the following Description of Illustrative Embodiments and claims in view of the accompanying figures of the drawing.

### BRIEF DESCRIPTION OF THE VIEWS OF THE DRAWING

FIG. 1 is a block diagram depicting a variety of components that may be included in various embodiments of the systems described herein.

FIG. 2 depicts various components of some embodiments of the systems described herein in use in determining the location of targeted tissue relative to a subject's tooth in the presence of a reference frame.

FIG. 3 depicts various components of some embodiments of the systems described herein in use in determining the location of targeted tissue relative to a subject's tooth without the use of a reference frame.

FIG. 4 depicts one embodiment of a mouth opening device that may be used in connection with the systems and methods described herein.

FIG. 5 is a block diagram depicting a variety of components that may be included in some embodiments of the systems described herein.

### DESCRIPTION OF ILLUSTRATIVE EMBODIMENTS

In the following description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

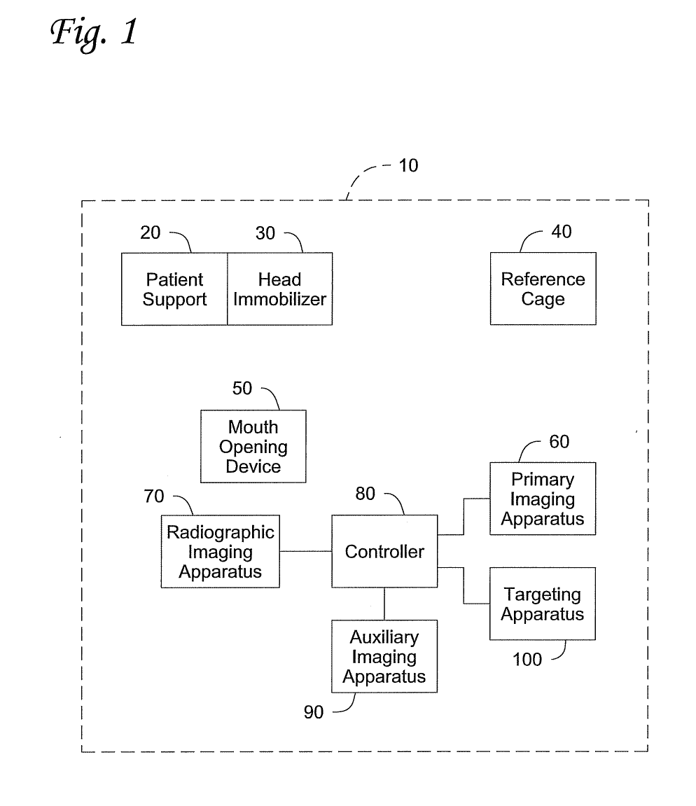

Components that may be provided in various embodiments of the systems described herein are depicted in the block diagram of FIG. 1. The components of some embodiments of the system 10 include, e.g., a subject support 20 and a head immobilizer 30. Although depicted as separate and discrete components, the subject support 20 and the head immobilizer 30 may be provided together as one integrated component.

The subject support 20 may take a variety of different fours including, but not limited to, a table, chair, bed, etc. Examples of some potentially useful subject supports may be described in, e.g., U.S. Pat. No. 5,207,223 (Adler); U.S. Pat. No. 5,427,097 (Depp); U.S. Pat. No. 5,528,651 (Leksell et al.); U.S. Pat. No. 6,778,850 (Adler et al.); U.S. Pat. No. 6,876,196 (Taulu et al.); U.S. Pat. No. 7,348,974 (Smith et al.); etc.

The head immobilizer 30 may take a variety of forms including, but not limited to, an inflatable air helmet, masks (e.g., thermoplastic masks, etc.), strap systems, cradles, etc. Regardless of its form, the head immobilizer is preferably capable of preventing movement of the head of a patient relative to the head immobilizer. Examples of some potentially suitable head restraints may be described in, e.g., U.S. Pat. No. 5,207,223 (Adler); U.S. Pat. No. 5,427,097 (Depp); U.S. Pat. No. 5,528,651 (Leksell et al.); U.S. Pat. No. 6,778,850 (Adler et al.); U.S. Pat. No. 6,876,196 (Taulu et al.); and U.S. Pat. No. 7,348,974 (Smith et al.); as well as in, e.g., U.S. Pat. No. 6,882,878 (Schmit et al.); U.S. Pat. No. 6,873,156 (Ferris et al.); U.S. Pat. No. 6,275,723 (Ferris et al.); U.S. Pat. No. 5,311,882 (Gagne); etc.

The system 10 may, in some embodiments, include a reference cage 40 that may preferably be capable of being fixedly attached to the subject support 20 and/or head immobilizer 30 such that the subject's tooth can be held stationary relative to the reference cage 40. As used herein, “fixedly attached” means that the components are attached in a manner that prevents relative movement between the fixedly attached components (through any appropriate techniques/structures, e.g., mechanical fasteners, adhesives, welding, etc.).

The reference cage 40 preferably defines a three-dimensional volume that can be imaged by both a radiological imaging system (e.g., a CT imaging system) that can also provide an image of the location of the targeted tissue as well as a non-radiological imaging system (e.g., optical cameras, etc.) that can obtain images of a subject's tooth and the reference cage 40.

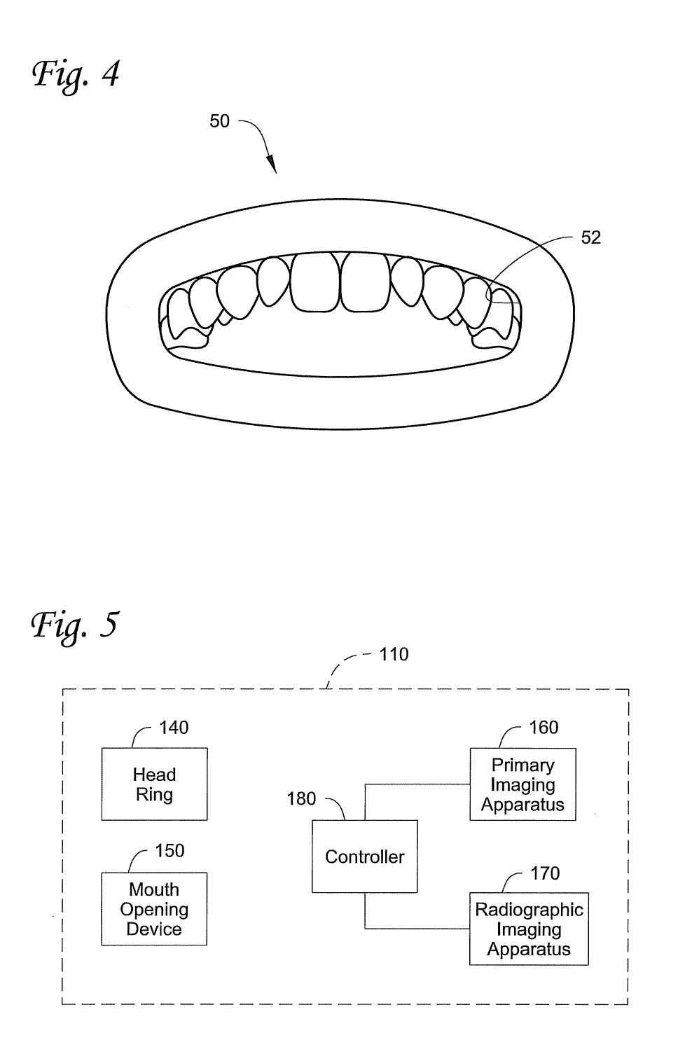

The system 10 may, in some embodiments, include an optional mouth opening device 50 that preferably functions to restrain the upper lip of a subject such that a non-radiological image of one or more upper teeth of the subject can be obtained. One embodiment of a mouth opening device 50 is depicted and described in connection with FIG. 4. The mouth opening device 50 may preferably be constructed of material that is substantially radiolucent for radiological imaging. Examples of some potentially useful mouth opening devices may be found in, e.g., U.S. Pat. No. 3,916,880 (Schroer); U.S. Pat. No. 7,300,400 (Brown); and others.

In some embodiments, the mouth opening devices may not be fixedly attached to the reference cages in the systems described herein. In such embodiments, the mouth opening devices and reference cases may move relative to each other such that the spatial relationships between the mouth opening devices and the reference cages is not fixed.

The system 10 may, in some embodiments, include a primary imaging apparatus 60 that can be used to obtain direct images of one or more teeth of a subject and the reference cage 40 (if present). The primary imaging apparatus 60 may preferably be a non-radiological imaging system that may also be a stereographic imaging system in the form of, e.g., a plurality of optical cameras. In some embodiments, the primary imaging apparatus 60 may take the form of two cameras (or other non-radiological imaging devices) for stereographic image capture and, optionally, a third camera/non-radiological imaging device and/or light source to provide for improved resolution in the direct images obtained by the primary imaging apparatus. The stereographic imaging systems described herein may preferably be designed to obtain the three-dimensional coordinates of selected points using two or more cameras (or one camera used in two or more locations) through camera calibration and triangulation.

As discussed herein, the primary imaging apparatus is used to obtain direct images of one or more of a subject's upper teeth. As used herein, a “direct image” may be obtained using light that is not considered to be radiological energy, with the light being in any suitable spectrum/wavelength, e.g., visible light (i.e., light visible to the normal human eye), infrared light, ultraviolet light, etc. In some instances, the primary imaging apparatus may be in the form of laser-based scanning system as opposed to an optical camera, etc. The primary imaging apparatus is preferably a non-radiological stereographic imaging apparatus.

The system 10 may, in some embodiments, include a radiological imaging apparatus 70 that can be used to obtain a radiological image of the targeted tissue and the reference cage 40 (when present). The radiological imaging apparatus may take the form of any suitable radiological imaging system capable of providing three-dimensional image data such as, e.g., a Computerized Tomography (CT) imaging system, a Magnetic Resonance Imaging (MRI) system, etc. As a result, a “radiologic image” as used herein is an image that is obtained using a radiological imaging system that is capable of providing three-dimensional image data while a “non-radiological image” is a “direct image”—that is, an image obtained through the use of electromagnetic energy that is not considered to be radiological image energy.

The system 10 may, in some embodiments, include a controller 80 that may take any suitable form, for example, the controller 80 may preferably include memory and a data processor. The data processor may be, e.g., an Application Specific Integrated Circuit (ASIC) state machine, a gate array, and/or may include a microprocessor. The controller 80 may also include components, etc. to operate other features not depicted in FIG. 1, e.g., sensors, alarms, etc.

The primary imaging apparatus 60 may, in some embodiments, be operably connected to a controller 80 such that images and/or image data that are obtained using the primary imaging apparatus 60 can be transmitted to the controller 80. The controller 80 may also preferably be operably connected to a radiological imaging apparatus 70 such that images and/or image data that are obtained using the radiological imaging apparatus 70 can be transmitted to the controller 80.

The controller 80 may, in some embodiments, include hardware and/or software such that the controller 80 can function to determine the location of targeted tissue relative to the reference cage 40 using image data from the radiological imaging apparatus 70; determine the location of one or more teeth relative to the reference cage 40 using the image data from the primary imaging apparatus 60; and determine the location of targeted tissue relative to one or more teeth using the determined location of the targeted tissue relative to the reference cage and the determined location of the teeth relative to the reference cage.

The system 10 may, in some embodiments, also include an auxiliary imaging apparatus 90 that may also be operably connected to the controller 80. The auxiliary imaging apparatus 90 may be used to, e.g., supply non-radiological (e.g., optical, etc.) images and/or image data of the primary imaging apparatus 60 to the controller 80. The auxiliary imaging apparatus 90 may preferably be in the form of a non-radiological stereographic imaging apparatus. The auxiliary image data can be used by the controller 80 to determine the location of the primary imaging apparatus 60 in a treatment room (e.g., an operating room or other space in which the subject is located during the targeting process). The controller 80 can then be used to determine the location of the targeted tissue in the treatment room using the determined location of the primary imaging apparatus 60 (which is used to determine the location of the tooth as discussed herein).

The system 10 may, in some embodiments, also include a targeting apparatus 100 that can be used to align targeted tissue with a predetermined location. Such a targeting apparatus may include, e.g., a system capable of moving the subject support 20 such that the targeted tissue would be positioned in a predetermined location in a treatment room where the targeted tissue could receive selected doses of radiation energy using one or more beams of radiation energy. In such a system, the subject support 20 may include a translational mechanism and/or rotational mechanism to move the subject support 20 within a treatment room and the targeting apparatus 100 is operably connected to the translational mechanism and/or rotational mechanism to move the subject support 20. Examples of some potentially useful targeting systems that may include different apparatus and control systems that can provide the positioning described herein may be described in, e.g., U.S. Pat. No. 5,207,223 (Adler); U.S. Pat. No. 5,427,097 (Depp); U.S. Pat. No. 5,528,651 (Leksell et al); U.S. Pat. No. 6,778,850 (Adler et al.); U.S. Pat. No. 6,876,196 (Taulu et al.); U.S. Pat. No. 7,348,974 (Smith et al.); etc.

Alternatively, the targeting apparatus 100 may be operably connected to the controller 80 such that the controller 80 and targeting apparatus 100 can be used to adjust the direction and/or emanation point of one or more beams of radiation emitted by a radiation beam generator such that the one or more beams are directed at the location of the targeted tissue as determined by the controller 80 using the image data. In other words, in some systems, the subject may be moved relative to a predetermined location in a treatment room at which radiation energy is delivered. In other systems, the beam or beams of radiation energy may be adjusted so that they are directed to the determined location of the targeted tissue. In still other systems, the subject may be moved and the beam or beams of radiation may be adjusted.

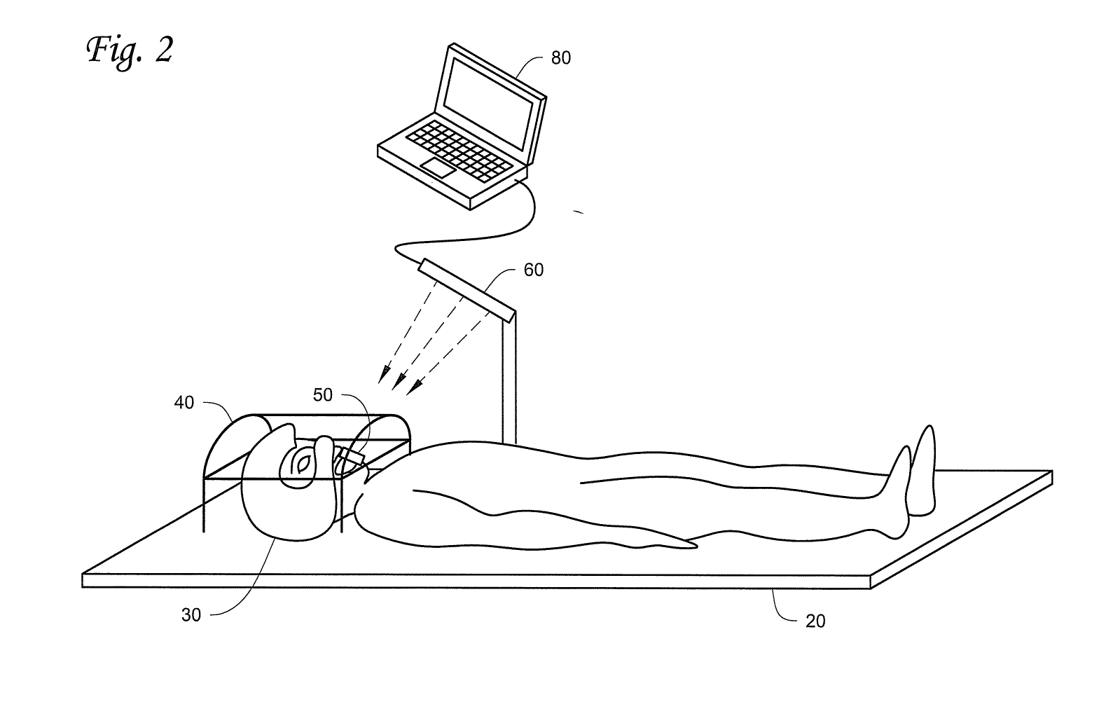

A diagram depicting one embodiment of a method and system in use to determine the location of the targeted tissue relative to one or more of a subject's teeth is seen in FIG. 2 in which a subject is located on a subject support 20, e.g., a table.

A mouth opening device 50 is placed in the subject's mouth such that the upper teeth are visible to the primary imaging apparatus 60. The mouth opening device 50 may be constructed of, e.g., a non-toxic plastic, and is designed in a way that the upper lip of the subject is restrained, supported (e.g., fully pulled out superiorly, etc.) to make as much area of the upper teeth visible to the primary imaging apparatus 60 such that a direct image can be obtained. The primary imaging apparatus 60 may, in some embodiments, be a stereographic imaging system designed to measure the three-dimensional (3D) coordinates of points from left-right image pair through camera calibration and triangulation.

The subject's head is immobilized with a head immobilizer 30 that may be in the form of, e.g., an inflatable air helmet or other structure/apparatus capable of preventing movement of a patient's head. A reference cage 40 that can provide a geometric frame of reference within a volume in which the subject's head is located may be attached to the subject support 20.

Radiological imaging may be performed of the subject's head and the reference cage using a radiological imaging apparatus (see FIG. 1) while the primary imaging apparatus 60 is taking direct images of the upper teeth of the subject and the reference cage 40. One or both forms of imaging may preferably be performed continuously and simultaneously and/or at discrete times (e.g., in a series of discrete images).

The controller 80 uses the radiological image data and the direct image data to determine the location of the targeted tissue relative to one or more teeth. The controller 80 does so by using: a) the radiological image data to determine the spatial relationship between the reference cage 40 and the targeted tissue; and b) the direct image data from the primary imaging apparatus 60 to determine the location of the teeth relative to the reference cage 40.

After the spatial relationship between the subject's teeth and the targeted tissue in the presence of the reference cage is determined, the location of the targeted tissue can be determined without the use of the reference cage. This mode may be particularly useful to determine the location of the targeted tissue using direct non-radiological imaging alone (i.e., additional radiological imaging is not required to determine the location of the targeted tissue).

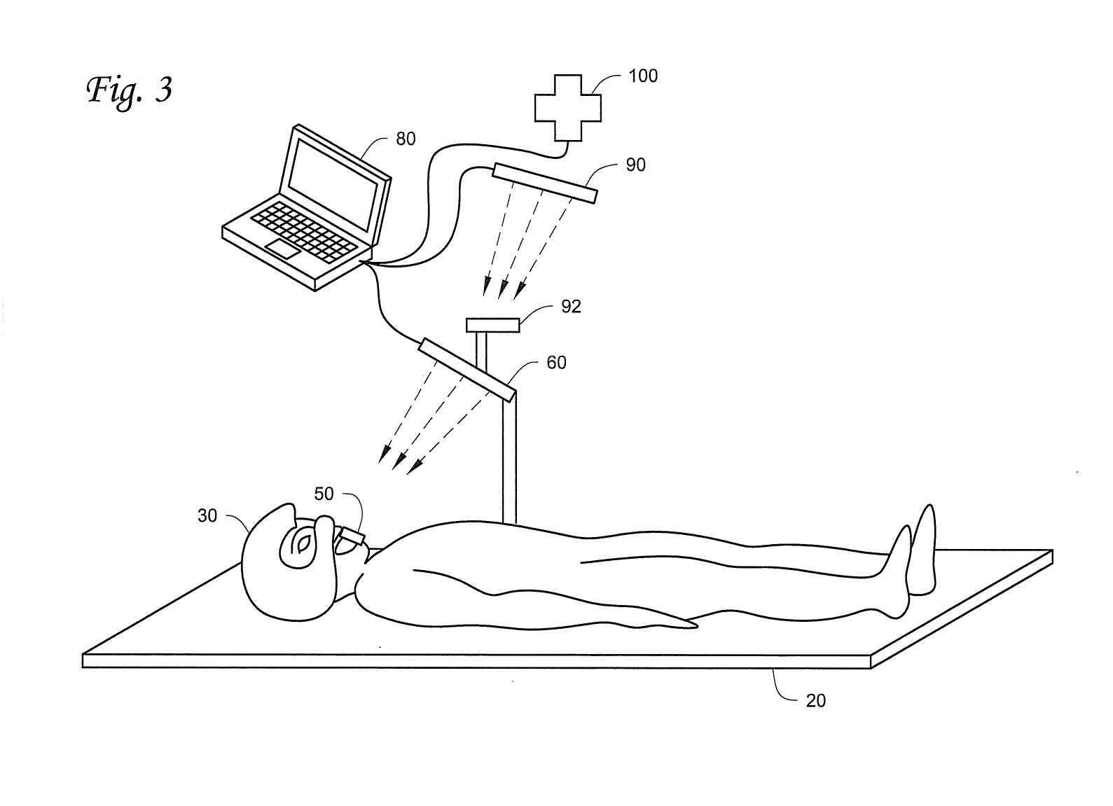

A diagram depicting one embodiment of a method and system in use to determine the location of the targeted tissue in a treatment room is seen in FIG. 3 in which a subject is located on a subject support 20, e.g., a table. In the method, the mouth opening device 50 is inserted into the subject's mouth to expose the upper teeth to the primary imaging apparatus 60. The subject's head is immobilized with a head immobilizer 30 that may be in the faun of, e.g., an inflatable air helmet, mask, etc.

As a part of the method, an auxiliary imaging apparatus 90 can be used to obtain non-radiological images of the primary imaging apparatus 60. The auxiliary images can be used to determine the position of the primary imaging apparatus 60 in the treatment room. The auxiliary imaging apparatus 90 is preferably located in a fixed position in a treatment room while the primary imaging apparatus 60 may be movable. As a result, the primary imaging apparatus may, in some embodiments, be insufficient to determine the location of targeted tissue within the treatment room.

The fixed auxiliary imaging apparatus 90 may, however, be used to determine the position of the primary imaging apparatus 60 in the treatment room. The auxiliary imaging apparatus 90 may use a marker 92 that is attached to (or otherwise associated with the position of) the primary imaging apparatus 60. The auxiliary imaging apparatus 90 can then determine the position of the marker 92 and, in turn, the position of the primary imaging apparatus 60.

With the position of the primary imaging apparatus 60 determined, the position of the subject's upper teeth in the treatment room and, thus, the position of the targeted tissue in the treatment room can be determined. Once the position of the targeted tissue in the treatment room is known, the controller 80 can be used to position the targeted tissue in a predetermined location (by, e.g., moving the subject) and/or adjust a radiation beam generator such that it directs one or more beams of radiation to the location of the targeted tissue as described herein.

Although depicted as singular devices, the primary imaging apparatus 60 and/or the auxiliary imaging apparatus 90 may be embodied in a single device (e.g., an optical camera) or, alternatively, may include more than one imaging device (e.g., two or more optical cameras, etc.).

FIG. 4 depicts one embodiment of a mouth opening device 50 that may be used in connection with the systems and methods described herein. The mouth opening device 50 preferably provides an opening 52 in which one or more of the front upper teeth (e.g., the incisors and, optionally, the canines) are exposed for non-radiological imaging using the primary imaging apparatus as described herein.

In some embodiments, the opening 52 in the mouth opening device 50 may have a selected shape (e.g., oval, trapezoidal, rectangular, circular, etc.) that is designed to assist with imaging and targeting using the imaging systems described herein, e.g., the primary imaging apparatus may be designed to acquire or seek the selected shape of the opening 52 as part of its target acquisition procedure. In some embodiments, the mouth opening device 50 may have a selected color (e.g., red, black, etc.), pattern of colors, selected portions having one or more selected colors, etc. that are designed to assist the primary imaging apparatus in its target acquisition procedure.

The mouth opening device 50 may be manufactured of any suitable material or combination of materials such as, e.g., polymers, glass, etc. It may be preferred that the mouth opening device 50 be substantially radiolucent to the radiological energy used during radiological imaging so that the mouth opening device 50 does not obscure the radiological images to a degree that substantially impairs their use for radiological imaging.

FIG. 5 depicts another potential embodiment of a system 110 in which a primary imaging apparatus 160, a radiological imaging apparatus 170, and a controller 180 can be used in a quality assurance mode to verify the accurate positioning of a stereotactic head ring 140 on the head of a subject using one or more direct images of one or more of the subject's upper teeth. The system may also include a mouth opening device 150, wherein the primary imaging apparatus 160 obtains a direct image of a tooth through the mouth opening device when the mouth opening device is in place within the mouth of a subject. The stereotactic head ring 140 defines a three-dimensional volume proximate a subject's head and includes mechanisms for attaching the head ring directly to the subject (as is conventionally known). The head ring 140 preferably includes radio-opaque material such that its position is detectable using radiological imaging and can be used to supply a frame of reference against which the location of targeted tissue can be determined.

As discussed in connection with this aspect, the conventional head ring is in the form of a stereographic ring that can be directly attached to or otherwise positioned in a stationary location on the head of the subject. A radiological imaging system is then used to determine the location of targeted tissue in the subject's head relative to the head ring. In use, however, these head rings may move or shift on the subject's head over time, thus potentially causing misalignment of the head ring and the targeted tissue, with corresponding misdirection of the radiation therapies being delivered.

The systems and methodologies described herein may, however, be adapted for use in assuring the accurate positioning of the head ring and, if necessary, providing for realignment of the head ring on the subject's head using one or more images of one or more of the upper teeth of the subject.

In one embodiment of such a methods, the location of a head ring relative to a tooth may be determined by attaching a stereotactic head ring to a head of a subject; exposing a tooth of the subject while the stereotactic head ring is attached to the head; obtaining a direct image of the tooth and the head ring using a primary imaging apparatus; and determining an original location of the tooth relative to the head ring using the direct image from the primary imaging apparatus.

With the original location of the head ring relative to the tooth known based on non-radiological direct images from the primary imaging apparatus, the method may further include, in some embodiments, determining the location of targeted tissue in the head of the subject relative to the head ring using a radiological imaging system.

In still other embodiments, the method may include adjusting the position of the head ring on the head of the subject such that the head ring and the tooth are in the original position where, for example, it is determined from subsequent imaging with a non-radiological imaging apparatus that the head ring has shifted and is no longer in the original location. With the head ring restored to its original location (relative to the tooth), further treatments may be more accurately directed to the targeted tissue as discussed elsewhere herein.

The complete disclosure of the patents, patent documents, and publications cited in the Background, the Detailed Description of Exemplary Embodiments, and elsewhere herein are incorporated by reference in their entirety as if each were individually incorporated (to the extent they do not conflict with the disclosure contained in this document).

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A stereotactic intracranial target localization guidance system comprising:
a subject support device comprising a head immobilizer;
a primary imaging apparatus, wherein the primary imaging apparatus comprises a non-radiological imaging system;
a reference cage defining a three-dimensional volume, wherein the reference cage comprises radio-opaque material; and
a controller operably connected to the primary imaging apparatus to receive direct image data from the primary imaging apparatus and wherein the controller receives radiological image data from a radiological imaging apparatus, wherein the controller is configured to:
determine the location of targeted tissue relative to the reference cage using the radiological image data from the radiological imaging apparatus;
determine the location of a selected upper tooth of a subject positioned in the subject support device relative to the reference cage using the direct image data from the primary imaging apparatus; and
determine the location of the targeted tissue relative to the selected upper tooth using the determined location of targeted tissue relative to the reference cage and the determined location of the selected upper tooth relative to the reference cage.

2. A system according to claim 1, wherein the reference cage is not attached to a mouth opening device.

3. A system according to claim 1, wherein the reference cage is fixedly attached to the subject support device.

4. A system according to claim 1, wherein the head immobilizer comprises an inflatable helmet.

5. A system according to claim 1, wherein the primary imaging apparatus comprises a stereographic imaging system.

6. A system according to claim 1, wherein the controller is operably connected to a radiation beam generator, wherein the controller is configured to direct the radiation beam generator to emit a beam of radiation at the targeted tissue based on the location of the tooth.

7. A system according to claim 1, wherein the subject support comprises a translational mechanism and/or rotational mechanism, and wherein the controller is operably connected to the translational mechanism and/or rotational mechanism of the subject support, and further wherein the controller is configured to operate the translational mechanism and/or rotational mechanism to position the determined location of the targeted tissue in a predetermined location.

8. A system according to claim 1, wherein the system comprises an auxiliary imaging apparatus located in a fixed position within a treatment room, wherein the auxiliary imaging apparatus obtains a non-radiological auxiliary image of the primary imaging apparatus, wherein the auxiliary imaging apparatus is operably connected to the controller, and wherein the controller is configured to:
determine the location of the primary imaging apparatus in the treatment room using auxiliary image data from the auxiliary imaging apparatus; and
determine the location of the targeted tissue within the treatment room using the determined location of the primary imaging apparatus.

9. A system according to claim 8, wherein the auxiliary imaging apparatus comprises a stereographic imaging system.

10. A system according to claim 8, wherein the controller is operably connected to a radiation beam generator, wherein the controller is configured to direct the radiation beam generator to emit a beam of radiation at the determined location of the targeted tissue.

11. A system according to claim 8, wherein the subject support comprises a translational mechanism and/or rotational mechanism, and wherein the controller is operably connected to the translational mechanism and/or rotational mechanism of the subject support, and further wherein the controller is configured to operate the translational mechanism and/or rotational mechanism to position the determined location of the targeted tissue in a predetermined location.

12. A method of determining the location of targeted tissue relative to a tooth, the method comprising:
positioning a head of a subject in a volume defined by an reference cage;
exposing a selected upper tooth of the subject while the head is positioned in the volume;
obtaining a direct image of the selected upper tooth using a non-radiological primary imaging apparatus;
obtaining a radiological image that includes the targeted tissue and the reference cage;
restraining the head of the subject in a fixed position relative to the reference cage while obtaining the direct image of the selected upper tooth using the primary imaging apparatus and while obtaining the radiological image that includes the targeted tissue and the reference cage;
determining the location of targeted tissue in the head relative to the reference cage using the radiological image;
determining the location of the selected upper tooth relative to the reference cage using the direct image from the primary imaging apparatus; and
determining the location of the targeted tissue relative to the selected upper tooth using the location of targeted tissue relative to the reference cage and the location of the selected upper tooth relative to the reference cage.

13. A method according to claim 12, wherein exposing the selected upper tooth comprises placing a mouth opening device in the mouth of the subject.

14. A method according to claim 13, wherein the mouth opening device is not fixedly attached to the reference cage.

15. A method according to claim 12, wherein the reference cage is fixedly attached to the subject support device.

16. A method according to claim 12, wherein restraining the head of the subject in a fixed position comprises positioning the head in an inflatable helmet and inflating the helmet.

17. A method according to claim 12, wherein the primary imaging apparatus comprises a stereographic imaging system.

18. A method according to claim 12, wherein the method further comprises:
obtaining an auxiliary image of the primary imaging apparatus using non-radiological auxiliary imaging apparatus located in a fixed position within a treatment room in which the reference cage is located;
determining the location of the primary imaging apparatus in the treatment room using auxiliary image data from the auxiliary imaging apparatus; and
determining the location of the targeted tissue within the treatment room based using the determined location of the primary imaging apparatus.

19. A method according to claim 18, wherein the auxiliary imaging apparatus comprises a stereographic imaging system.

20. A method according to claim 18, wherein the method further comprises adjusting the position of the subject support to place the targeted tissue in a predetermined location within the treatment room.

21. A method according to claim 18, wherein the method further comprises emitting a beam of radiation at the determined location of the targeted tissue.

22. A method according to claim 12, wherein the method further comprises adjusting the position of the subject support to place the targeted tissue in a predetermined location within a treatment room.

23. A method according to claim 12, the method further comprising emitting a beam of radiation at the determined location of the targeted tissue.

24. A stereotactic intracranial target localization guidance system comprising:
a primary imaging apparatus, wherein the primary imaging apparatus comprises a non-radiological imaging system;
a stereotactic head ring defining a three-dimensional volume proximate a subject's head, wherein the head ring comprises mechanisms for attaching the head ring directly to the subject, and wherein the head ring comprises radio-opaque material; and
a controller operably connected to the primary imaging apparatus to receive direct image data from the primary imaging apparatus, wherein the controller is configured to determine an original location of the head ring relative to the tooth using the direct image data from the primary imaging apparatus.

25. A system according to claim 1, wherein the system further comprises a subject support device that optionally comprises a head immobilizer.

26. A method of determining a location of a head ring relative to a tooth, the method comprising:
attaching a stereotactic head ring to a head of a subject;
exposing a selected upper tooth of the subject while the stereotactic head ring is attached to the head;
obtaining a direct image of the selected upper tooth and the head ring using a non-radiological primary imaging apparatus; and
determining an original location of the selected upper tooth relative to the head ring using the direct image from the primary imaging apparatus.

27. A method according to claim 26, wherein the method further comprises determining the location of targeted tissue in the head of the subject relative to the head ring using a radiological imaging system after determining the original location of the selected upper tooth relative to the head ring using the primary imaging apparatus.

28. A method according to claim 26, wherein the method comprises adjusting the position of the head ring on the head of the subject such that the head ring and the selected upper tooth are in the original position.
